(19) United States
(12) Patent Application Publication
(10) Pub. No.: US 2004/0009926 A1
(43) Pub. Date: Jan. 15, 2004

(54) Carnitine in the treatment of depression

(75) Inventors: Jay W. Pettegrew, Pittsburgh, PA (US); Samuel Gershon, Fox Chapel, PA (US)

Correspondence Address: NIXON & VANDERHYE, PC, 1100 N GLEBE ROAD, 8TH FLOOR, ARLINGTON, VA, 22201-4714, US

(73) Assignee: Sigma-Tau Industrie Farmaceutiche Riunite S.p.A.

(21) Appl. No.: 10/359,560

(22) Filed: Feb. 7, 2003

Related U.S. Application Data
(60) Provisional application No. 60/354,323, filed on Feb. 7, 2002.

(51) Int. Cl. A61K 31/7024; A61K 31/22; A61K 31/66

(52) U.S. Cl. 514/23; 514/550; 514/129

(57) ABSTRACT

Geriatric depression is treated with L-carnitine or an alkanoyl L-carnitine, desirably acetyl L-carnitine thereby avoiding unwanted side-effects exhibited by conventional antidepressant agents.

### CROSS-REFERENCE TO RELATED APPLICATION

This application claims benefit under 35 U.S.C. §119(e) of Provisional Application Serial No. 60/354,323 filed Feb. 7, 2002.

This invention relates to the treatment of depression, particularly geriatric subjects.

### BACKGROUND OF THE INVENTION

The clinical response to antidepressant treatment in later life follows a variable temporal response, with a median time to remission of 12 weeks. Newer antidepressants still demonstrate a disturbing side-effect profile in this fragile patient population. Thus, there is a need for the development of newer antidepressants. One such candidate is acetyl-L-carnitine, a molecule that is naturally present in human brain demonstrating only few side effects.

Seven parallel, double-blind, placebo-controlled studies have examined ALCAR efficacy in various forms of geriatric depression. Phosphorus magnetic resonance spectroscopy (³¹P MRS) directly provides information on membrane phospholipid and high-energy phosphate metabolism in defined, localized brain regions. Although in vivo ³¹P MRS studies in major depression are limited, there is evidence of altered high-energy phosphate and membrane phospholipid metabolism in the prefrontal and basal ganglia regions. Increased levels of precursors of membrane phospholipids [i.e., increased phosphomonoesters (PME) levels] in the frontal lobe of major depressed subjects compared to controls was reported. Other researchers also observed higher PME levels in bipolar subjects in their depressive phase compared with the euthymic state. In terms of high-energy phosphates, reduced levels of adenosine triphosphate (ATP) have been observed in both the frontal and basal ganglia of major depressed subjects. The level of the high-energy phosphate buffer, phosphocreatine (PCr), was lower in severely depressed subjects compared with mildly depressed subjects. Accordingly, the relationship between membrane phospholipid and high-energy phosphate metabolism as assessments of beneficial results in the treatment of depression are recognized.

### BRIEF DESCRIPTION OF THE DRAWINGS

FIG. 1(a) is a graph showing the correlation of PME(s−τ_{c}) levels from the prefrontal region with HDRS scores for both depressed patients (● subject #1; ♦ subject #2);

FIG. 1(b) is a graph showing the correlation of PCr levels from the prefrontal region with HDRS scores for both depressed patients (● subject #1; ♦ subject #2);

FIG. 2(a) is a graph showing PME(s−τ_{c}) and PCr levels in the a) prefrontal region of the two depressed patients (● subject #1; ♦ subject #2) and normal controls (O, n=6) at baseline and at 6 and 12 weeks follow up. The control values include mean±SD;

FIG. 2(b) is a graph showing PME(s−τ_{c}) and PCr levels in the basal ganglia region of the two depressed patients (● subject #1; ♦ subject #2) and normal controls (O, n=6) at baseline and at 6 and 12 weeks follow up. The control values include mean±SD;

FIG. 3(a) is a phosphorous magnetic resonance spectroscopic image showing the Z-scores of the two depressed subjects compared with controls at entry and 12 weeks for PME(s−τ_{c}) metabolite levels for those regions with significant differences. The intensity of the color is scaled to the z-score (mean difference/SD) given on the scale below the image. Z-scores for PME(s−τ_{c}) and PCr levels in the frontal region exceed 3.0 and 2.0, respectively; and

FIG. 3(b) ) is a phosphorous magnetic resonance spectroscopic image showing the Z-scores of the two depressed subjects compared with controls at entry and 12 weeks for PCr metabolite levels for those regions with significant differences. The intensity of the color is scaled to the z-score (mean difference/SD) given on the scale below the image. Z-scores for PME(s−τ_{c}) and PCr levels in the frontal region exceed 2.0 and 2.0, respectively.

### DESCRIPTION OF THE INVENTION

In the context of the invention described herein, it has been found, in an entirely unexpected way, that the use of a therapeutically effective amount of L-carnitine or of an alkanoyl L-carnitine, in which the linear or branched alkanoyl has 2-8 carbon atoms, or one of its pharmacologically acceptable salts, is beneficial to depressed subjects, particularly in geriatric patients, without a disturbing side-effect profile exhibited by traditional antidepressants and improving the quality of life itself in the subjects treated, whether human subjects or animals.

Carnitines in general are compounds of formula (I):

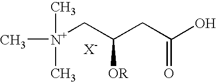

where R is hydrogen or an alkanoyl group with 2 to 8 carbon atoms, and X− represents the anion of a pharmaceutically acceptable salt.

The invention described herein includes both the administration of L-carnitine or an alkanoyl L-carnitine or one of its pharmacologically acceptable salts of formula (I) in the treatment of depression, and pharmaceutical compositions, which can be administered orally, parenterally or nasally, including controlled-release forms. Preferably, the alkanoyl L-carnitine is selected from the group consisting of acetyl L-carnitine (hereinafter abbreviated to ALC or ALCAR), propionyl L-carnitine (hereinafter abbreviated to PLC), butyryl L-carnitine, valeryl L-carnitine and isovaleryl L-carnitine, or one of their pharmacologically acceptable salts. The ones preferred are acetyl L-carnitine, propionyl L-carnitine and butyryl L-carnitine. The most preferred is acetyl L-carnitine.

What is meant by a pharmacologically acceptable salt of L-carnitine or of an alkanoyl L-carnitine is any salt of the latter with an acid that does not give rise to toxic or side effects. These acids are well known to pharmacologists and to experts in pharmaceutical technology.

Examples of pharmacologically acceptable salts of L-carnitine or of the alkanoyl L-carnitines, though not exclusively these, are chloride; bromide; iodide; aspartate; acid aspartate; citrate; acid citrate; tartrate; acid tartrate; phosphate; acid phosphate; fumarate; acid fumarate; glycerophosphate; glucose phosphate; lactate; maleate; acid maleate; mucate; orotate, oxalate; acid oxalate; sulphate; acid sulphate; trichloroacetate; trifluoroacetate; methane sulphonate; pamoate and acid pamoate.

As used herein, a geriatric subject is an individual 65 years of age or older. See The Merck Manual, 15^{th} edition (1987) p. 2389.

One preferred form of daily dosing of L-carnitine or alkanoyl L-carnitine for clinical use is a composition comprising an amount of L-carnitine or an alkanoyl L-carnitine, preferably acetyl or propionyl L-carnitine, equivalent to 0.1 to 3 g, and preferably 0.5 to 3 g per day.

### DESCRIPTION OF THE PREFERRED EMBODIMENTS

Phosphorus magnetic resonance spectroscopic imaging (³¹P MRSI) analysis of two depressed elderly subjects treated with ALCAR for 12 weeks are compared with those of six normal non-demented, non-depressed subjects.

A twelve-week, open, clinical, ³¹P MRSI study design was used to examine the possible effects of ALCAR on brain metabolism and depressive symptomatology in non-demented geriatric major depressive disorder (NDG-MDD). Two depressed, non-demented [Folstein Mini-Mental State Exam (MMSE)>24)] male subjects, 70 and 80 years old, were compared with six age, social-economic status, and medically matched non-demented controls (all male, mean age of 73.6±3.6 years, range 69.7-78.2 years). The two elderly depressed subjects completed baseline Structural Clinical Interview of DSM-IV (SCID) I/P version 2.0, HDRS (17 item), MMSE, UKU Side Effect Rating Scale (UKU), and Cumulative Illness Rating Scale (CIRS) to assess medical burden, baseline physical, ECG, and, laboratory tests for hematology, urine analysis, immunopathology, and blood chemistry. Follow-up visits for the depressed subjects were done every other week for 12 weeks. Efficacy (psychiatric evaluation) was assessed by changes in the HDRS which was performed at baseline and every other week for 12 weeks along with secondary measures (MMSE; CIRS; and UKU), whereas the CIRS was performed at baseline, 6, and 12 weeks. Physical examinations and EKGs were performed at baseline, 6, and 12 weeks. The baseline MR evaluation was scheduled and completed prior to the administration of ALCAR. Follow-up MR evaluations were at 6 and 12 weeks. Acetyl-L-carnitine was administered in the form of oral tablets containing 590 mg of acetyl-L-carnitine hydrochloride (500 mg acetyl-L-carnitine). The dosage regimen was fixed at three grams of acetyl-L-carnitine given two tablets three times a day for 12 weeks.

³¹P MRSI acquisition—A custom built, doubly tuned transmit/receive volume head coil was used to acquire the ¹H MRI and 2D ³¹P MRSI data on a GE Signa 1.5 T whole body MR imager. First, sets of axial and sagittal scout MR images were collected. The 30 mm thick MRSI slice was positioned parallel with the anterior commisure-posterior commisure line to include the right and left prefrontal, basal ganglia, superior temporal, inferior parietal, occipital, and centrum semiovale regions. A self-refocused spin echo pulse sequence with an effective flip range of 60° and an echo time of 2.5 ms, was used to acquire the ³¹P MRSI (360 mm field of view, 30 mm slice thickness, 8×8 phase encoding steps [45×45×30 mm³ nominal voxel dimensions], 2 s TR, 1024 data points, 4.0 kHz spectral bandwidth and 16 NEX).

MRSI post-processing and quantification—To optimize the right and left voxel positions for the six regions, the 8×8 ³¹P grid was shifted with respect to the anatomical MRI and a mild spatial apodization (i.e., Fermi window with 90% diameter and 5% transition width) was applied prior to the inverse Fourier transform. The remaining processing steps were 100% automated. A 5 Hz exponential apodization was applied and the PME, phosphodiester (PDE), PCr, α-, γ-, and β-ATP, and inorganic orthophosphate (Pi), were modeled in the time domain with exponentially damped sinusoids and by omitting the first 2.75 ms of the free induction decay (FID) using the Marquardt-Levenberg algorithm. This approach ensured that the PME and PDE resonances primarily reflected the freely mobile, short correlation time (s−τ_{c}), water soluble PME(s−τ_{c}) and PDE(s−τ_{c}) metabolites without the influence of relatively broad underlying signals within the PME and PDE spectral region. The PME(s−τ_{c}) (i.e., phosphoethanolamine, phosphocholine, and inositol-1-phosphate) are predominantly building blocks of phospholipids and therefore, the relative concentrations of these metabolites are a measure of the active synthesis of membranes; the PDE(s−τ_{c}) (i.e., glycerophosphocholine and glycerophosphoethanolamine) are major products of membrane degradation. To obtain intermediate correlation time (i−τ_{c}) components within the PME and PDE spectral region, the FIDs were modeled a second time but with omitting the first 0.75 ms of the FID and then taking the difference between the PME and PDE amplitudes of the two modeled results. PME(i−τ_{c}) moieties include less mobile molecules such as phosphorylated proteins and PMEs that are tightly coupled (in terms of MRS) to macromolecules [i.e., PMEs inserting into membrane phospholipids. PDE(i−τ_{c}) moieties include less mobile PDEs that are part of small membrane phospholipid structures such as micelles, synaptic vesicles, and transport/secretory vesicles and PDE moieties coupled to larger molecular structures (i.e., PDEs inserting into membrane phospholipid structures. The right/left side effect was eliminated by averaging the signal from the two voxels, prior to fitting (which included correcting for phase and resonance frequency). Additionally, metabolite levels are expressed as a mole % relative to the total ³¹ P signal.

The statistical analysis was done using the Statview (SAS Institute, Inc.) software package. The pearson t correlation test used to correlate between variables.

The two elderly depressed subjects were diagnosed with MDD according to DSM-IV criteria. No previous antidepressant medications were taken by the subjects in the three months prior to the study. Subject #1 has baseline, 6 and 12 week HDRS scores of 15, 1 and 0 and subject #2 had scores of 20, 17, and 3, respectively. Thus both depressed subjects were clinically improved at endpoint, fulfilling criteria for remission (HDRS<8). Medical conditions diagnosed in the depressed subjects included s/p knee arthroscopy, s/p cervical disk removal, hearing loss and benign prostatic hypertrophy in subject #1 and benign prostatic hypertrophy in subject #2. No clinically significant abnormalities were found in the laboratory exams and EKG of either depressed subject. Baseline, 6, and 12 weeks CIRS were 7, 6, and 5 for subject #1; and 4, 4, and 2 for subject #2, respectively. The change reflects the improvement of depressive symptomatology. Side effects from ALCAR treatment were mild and included dry mouth in subject #1 and a slight increase in perspiration in subject #2.

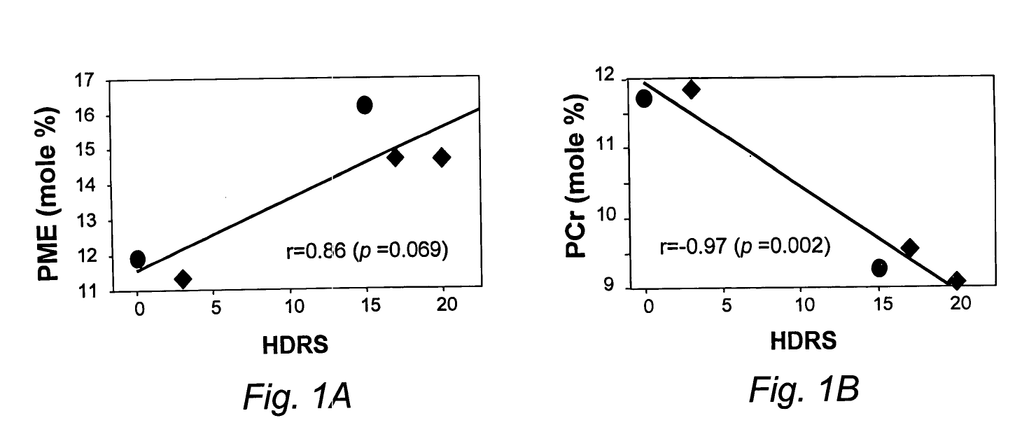

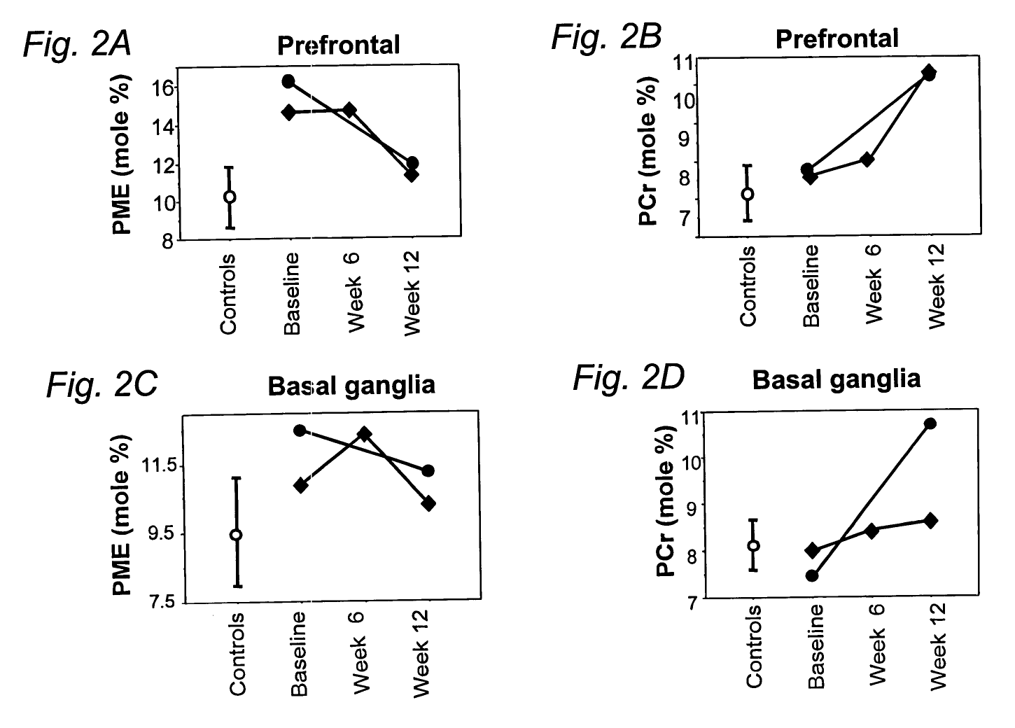

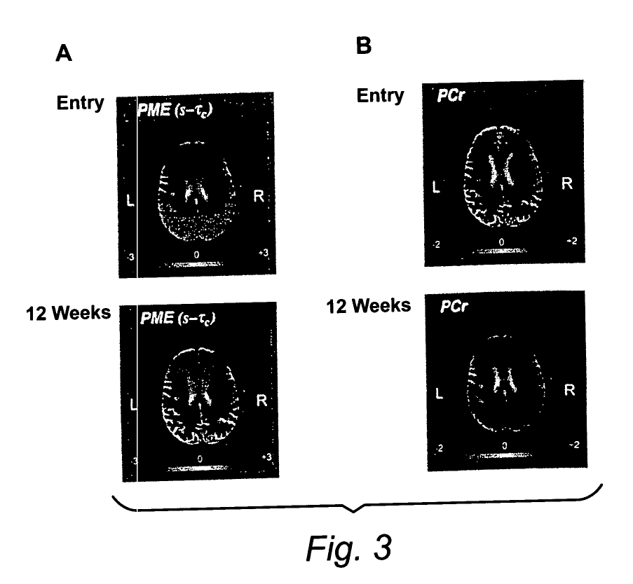

FIG. 1 shows the correlation of PME(s−τ_{c}) (r=0.86, p=0.069 and PCr (r=0.97, p=0.002) levels from the prefrontal region with HDRS scores for both depressed subjects. FIG. 2 illustrates the prefrontal and basal ganglia PCr and PME(s−τ_{c}) levels at baseline, 6 and 12 weeks for the two depressed subjects and the mean PCr and PME(s−τ_{c}) levels for the six normal controls. Unfortunately, the 6 week ³¹P MRSI session for subject #1 produced poor quality, unacceptable data and this time point is missing from the graphs. Baseline prefrontal PME(s−τ_{c}) levels in the depressed subjects were 1.5 to 2.0 SD higher than the mean of the controls and this increase was normalized with ALCAR treatment. Both depressed subjects had prefrontal PCr levels one SD higher than the mean of controls and ALCAR treatment further increased PCr levels by 27% and 31%, respectively. Similar changes in PME(s−τ_{c}) and PCr levels also were observed in the basal ganglia region (FIG. 2), but these metabolite levels did not correlate with HDRS scores. Although the most marked changes occur in the prefrontal region, z-score plots of the significant PME(s−τ_{c}) and PCr changes between depressed subjects and controls illustrates the other brain regions also undergo changes with ALCAR treatment. FIG. 3 demonstrates that compared with normal subjects, the two untreated depressed subjects at baseline had increased levels of PME(s−τ_{c}) in the prefrontal region (p=0.006). After 12 weeks of ALCAR treatment, the PME(s−τ_{c}) are normalized in the prefrontal regions but elevated in the superior temporal regions (p=0.05. In addition, PCr levels are elevated in the prefrontal (p=0.001), basal ganglia (p=0.022), and occipital (p=0.027 regions after 12 weeks of ALCAR treatment. There were no significant changes in the other metabolite levels.

While not wishing to be bound by any particular theory, the above findings suggest that beneficial clinical effects of acetyl-L-carnitine appear to be associated with changes in brain prefrontal PME(s−τ_{c}) and PCr levels. In the prefrontal region, the depressed subjects compared with controls after 12 weeks of ALCAR treatment show normalization of PME(s−τ_{c}) and elevation of PCr levels.

The PME(s−τ_{c}) resonance is predominantly composed of phosphocholine, phosphoethanolamine and inositol-1-phosphate which are precursors in membrane phospholipid metabolism. The increased PME(s−τ_{c}) in depression, as also observed by others is not fully understood and will require further study. ALCAR treatment seems to restore PME(s−τ_{c}) levels to normal and there was a trend for the decreasing PME levels to correlate with clinical improvement. In the prefrontal region, twelve weeks of ALCAR treatment also elevated PCr, a high-energy phosphate metabolite which is an immediate precursor of ATP.

Compared with the control group, similar findings were observed for basal ganglia PME(s−τ_{c}) and PCr levels, but the metabolite levels did not correlate with HDRS scores. This may be due to the small number of depressed patients analyzed. Other brain regions may be affected by depression and these changes may be altered by ALCAR treatment (FIG. 3).

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

### REFERENCES

1. Reynolds C F, Frank E, Perel J, Mazumdar S, Kupfer D J. Maintenance therapies for late-life recurrent major depression: Research and review circa 1995. Int Psychogeriatr 1995; 7: 27-40.

2. Hsu J H, Shen W W. Male sexual side effects associated with antidepressants: a descriptive clinical study of 32 patients. Int J Psychiatry Med 1995; 25: 191-201.

3. Nelva A, Guy C, Tardy-Poncet B, Beyens M N, Ratrema M, Benedetti C, Ollagnier M. [Hemorrhagic syndromes related to selective serotonin reuptake inhibitor (SSRI) antidepressants. Seven case reports and review of the literature] [French]. Revue De Medecine Interne 2000; 21: 152-160.

4. Bremer J. Carnitine—metabolism and functions. Physiol Rev 1983; 63: 1420-1480.

5. Pettegrew J W, Levine J, McClure R J. Acetyl-L-carnitine physical-chemical, metabolic, and therapeutic properties: Relevance for its mode of action in Alzheimer's disease and geriatric depression. Mol Psychiatry 2000; 5: 616-632.

6. Kato T, Inubushi T, Kato. Magnetic resonance spectroscopy in affective disorders. J Neuropsychiatry Clin Neurosci 1998; 10: 133-147.

7. Moore C M, Frederick B B, Renshaw P F. Brain biochemistry using magnetic resonance spectroscopy: relevance to psychiatric illness in the elderly. J Geriatr Psychiatry Neurol 1999; 12: 107-117.

1. A method for treating depression in a subject comprising the administration to a subject suffering from depression an effective, an effective amount of a carnitine selected from L-carnitine or alkanoyl L-carnitine or of a pharmaceutically acceptable salt thereof.

2. The method of claim 1, wherein the alkanoyl L-carnitine is selected from the group consisting of acetyl L-carnitine, valeryl L-carnitine, isovaleryl L-carnitine and butyryl L-carnitine or their pharmacologically acceptable salts or mixtures thereof.

3. The method of claim 1, wherein the pharmacologically acceptable salt is selected from the group consisting of chloride; bromide; iodide; aspartate, acid aspartate; citrate, acid citrate; tartrate; phosphate, acid phosphate; fumarate, acid fumarate; glycerophosphate; glucose phosphate; lactate; maleate, acid maleate; mucate; orotate; oxalate; acid oxalate; sulphate, acid sulphate; trichloroacetate; trifluoroacetate and methane sulphonate.

4. The method according to claim 1, wherein said administration is in the form of a composition comprising said carnitine or a pharmaceutically acceptable salt thereof in combination with a pharmaceutically acceptable excipient and/or vehicle.

5. The method according to claim 1, wherein 0.1 to 3 g/day of the carnitine or of an equivalent amount of a pharmaceutically acceptable salt thereof are administered.

6. The method according to claim 1, wherein acetyl L-carnitine is administered as a pharmacologically acceptable salt selected from the group consisting of chloride, bromide, orotate, acid aspartate, acid citrate, acid phosphate, fumarate and acid fumarate, maleate and acid maleate, acid oxalate, acid sulphate, glucose phosphate, tartrate and acid tartrate.

7. The method of claim 1 wherein the carnitine is administered in the form of a dietary supplement.

8. The method of claim 1, wherein the carnitine is administered orally, parenterally, rectally, sublingually or transdermally, in the form of a medicament.
